(19) United States
(12) Patent Application Publication
(10) Pub. No.: US 2025/0025524 A1
(43) Pub. Date: Jan. 23, 2025

(54) HERBAL COMPOSITION AND METHOD FOR TREATING OR PREVENTING ACUTE OR CHRONIC PAIN

(71) Applicants: Jayesh Chaudhary, Mumbai (IN); Shalini Srivastava, Mumbai (IN); Rekha Patel, Mumbai (IN); Ashish Chinchansure, Mumbai (IN)

(72) Inventors: Jayesh Chaudhary, Mumbai (IN); Shalini Srivastava, Mumbai (IN); Rekha Patel, Mumbai (IN); Ashish Chinchansure, Mumbai (IN)

(21) Appl. No.: 18/222,533

(22) Filed: Jul. 17, 2023

(51) Int. Cl. A61K 36/53; A61K 9/00; A61K 36/9068; A61K 47/02; A61P 19/02; A61P 29/00

(52) U.S. Cl. CPC A61K 36/53; A61K 9/0053; A61K 36/9068; A61K 47/02; A61P 19/02; A61P 29/00; A61K 2236/15; A61K 2236/331; A61K 2236/37; A61K 2236/39; A61K 2236/51

(57) ABSTRACT

The present disclosure discloses a herbal composition comprising herbal composition comprising a Vitex negundo aqueous leaf extract powder; a supercritical CO₂ Zingiber officinale rhizome oleoresin; and pharmaceutical acceptable carrier wherein the ratio of Vitex negundo aqueous leaf extract powder to supercritical CO₂ Zingiber officinale rhizome oleoresin to pharmaceutical acceptable carrier is in the range of 80:20:0 to 89:9:2. The present disclosure also discloses a process of preparation of a herbal composition. The present herbal composition is clinically proven effective for reducing acute or chronic musculoskeletal pain, alleviating physical-activity induced joint pain such as mechanical loads on joints, reducing exercise-induced knee joint pain and chronic non-specific low back pain and related dysfunction.

### FIELD OF THE INVENTION

The present disclosure pertains to a herbal composition comprising standardized Vitex negundo leaf extract and Zingiber officinale rhizome oleoresin. Particularly to fill the gap for a safe and quick-acting pain-relieving herbal composition to alleviate challenges of side effects with conventional pain-relieving products like NSAIDs.

### BACKGROUND OF THE INVENTION

Increased awareness about the occurrence of side effects, and small to moderate, primarily short-term effects of synthetic analgesics have been cause for search for pain management.

Several nutraceuticals are available in the market these days for improving joint health, including collagen supplements, glucosamine hydrochloride, chondroitin sulfate, hyaluronic acid, methylsulfonylmethane, vitamin D, and likewise. These supplements help in reducing joint pain by reducing inflammation or by protecting the cartilage in the joints. However, their onset of action is slow and the research into these supplements tends to produce mixed results in terms of magnitude of effect to several methodological issues.

Clinical studies have supported the efficacy of herbal or plant-based ingredients in managing joint pain. However, most of them have been studied for their effect in chronic pain conditions such as osteoarthritis, while very few like Curcumin, Capsaicin, and Ginger have been studied for their role in acute pain conditions. This situation has created a need for an evidence-based fast-acting option for managing joint pain and thereby improving physical activity.

Thus, there remains an unmet need to provide an herbal composition which can fill the gap of a safe and quick-acting pain-relieving product that can be used by any group of individuals without fearing the side effects.

### OBJECTS OF THE INVENTION

An object of the present disclosure is to provide an herbal composition comprising Vitex negundo aqueous leaf extract powder, Zingiber officinalis rhizome oleoresin and with or without pharmaceutical acceptable carrier.

Another object of the present disclosure discloses a fast-acting method for treating or preventing acute or chronic musculoskeletal pain, physical-activity induced joint pain and related dysfunction.

Another object of the present disclosure discloses a method for treating or preventing osteoarthritis

Another object of the present disclosure is to fill the gap of a safe and quick-acting pain-relieving product.

### SUMMARY OF THE INVENTION

This summary is provided to introduce a selection of concepts in a simplified form that are further described below in Detailed Description section. This summary is not intended to identify key features or essential features of the claimed subject matter, nor is it intended to be used as an aid in determining the scope of the claimed subject matter.

An aspect according to one or more embodiments of the present disclosure is to provide an herbal composition comprising a Vitex negundo aqueous leaf extract powder; Zingiber officinale rhizome oleoresin; and with or without pharmaceutical acceptable carrier, wherein the ratio of Vitex negundo aqueous leaf extract powder to Zingiber officinale rhizome oleoresin to pharmaceutical acceptable carrier is in the range of 80:20:0 to 89:9:2.

Another aspect according to one or more embodiments of the present disclosure is to provide a fast acting method for treating or preventing acute or chronic musculoskeletal pain, physical-activity induced joint pain and related dysfunction comprising administering an effective amount of the herbal composition comprising a Vitex negundo aqueous leaf extract powder; a Zingiber officinale rhizome oleoresin; and with or without pharmaceutical acceptable carrier to a subject in need of such treatment

Another aspect according to one or more embodiments of the present disclosure is to provide a method for improving the range of motion of stiff joints comprising administering an effective amount of the herbal composition comprising a Vitex negundo aqueous leaf extract powder; a Zingiber officinale rhizome oleoresin; and with or without pharmaceutical acceptable carrier to a subject in need of such treatment

Other aspects of the invention will be set forth in the description which follows, and in part will be apparent from the description, or may be learnt by the practice of the invention.

### BRIEF DESCRIPTION OF THE DRAWINGS

Characteristics and advantages of the subject matter as disclosed in the present disclosure will become clearer from the detailed description of an embodiment thereof, with reference to the attached drawing, given purely by way of an example, in which:

FIG. 1 is an effect of study products that is product comprising an herbal composition in accordance with the present disclosure in comparison with a placebo product on pain intensity.

FIG. 2 is an effect of study products on work productivity.

FIG. 3 is an effect of study products on Insomnia Severity Index.

FIG. 4 is a sustained effect of study products on functional activity.

FIG. 5 is a sustained effect of the study products on the pain intensity.

FIG. 6 is the percentage of participants having perceptible pain relief at 2 hours post study products administration.

FIG. 7 is the No. of participants who achieved meaningful pain relief post administration of study products.

FIG. 8 is a mean pain intensity difference (mm) at Day1 due to effect of study products.

FIG. 9 is the percentage of responders and non-responders of study products on day 5.

FIG. 10 is the effect of study products on pain VAS scores at various timepoints during day 1 and day 5.

FIG. 11 is the percentage of participants with MPR and time taken to achieve MPR with various study products.

FIG. 12 is the change (Median) in VAS score due to study products.

FIG. 13 is the time-weighted sum of pain intensity difference (mm) of various study products.

FIG. 14 is the BPI pain severity scores of various study products.

FIG. 15 is the BPI interference scores of various study products.

### DETAILED DESCRIPTION OF THE INVENTION

The following is a detailed description of embodiments of the disclosure. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In some embodiments, numbers have been used for quantifying weights, percentages, ratios, and so forth, to describe and claim certain embodiments of the invention and are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Various terms as used herein are shown below. To the extent a term used in a claim is not defined below, it should be given the broadest definition persons in the pertinent art have given that term as reflected in printed publications and issued patents at the time of filing.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Unless the context requires otherwise, throughout the specification which follow, the word “comprise” and variations thereof, such as, “comprises” and “comprising” are to be construed in an open, inclusive sense that is as “including, but not limited to.”

The recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified.

The description that follows, and the embodiments described therein, is provided by way of illustration of an example, or examples, of particular embodiments of the principles and aspects of the present disclosure. These examples are provided for the purposes of explanation, and not of limitation, of those principles and of the disclosure.

It should also be appreciated that the present disclosure can be implemented in numerous ways, including as a system, a method or a device. In this specification, these implementations, or any other form that the invention may take, may be referred to as processes. In general, the order of the steps of the disclosed processes may be altered within the scope of the invention.

The headings and abstract of the invention provided herein are for convenience only and do not interpret the scope or meaning of the embodiments.

The following discussion provides many example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

According to an embodiment, the present disclosure provides an herbal composition comprising a Vitex negundo aqueous leaf extract powder; Zingiber officinale rhizome oleoresin; and with or without pharmaceutical acceptable carrier.

In one embodiment, the present disclosure provides an herbal composition comprising from about 80% to about 89% w/w of a Vitex negundo aqueous leaf extract powder; from about 20% to about 9% of Zingiber officinale rhizome oleoresin and from 0% to 2% of Silicon dioxide as an anticaking agent.

In one embodiment, the ratio of Vitex negundo aqueous leaf extract powder to Zingiber officinale rhizome oleoresin to pharmaceutical acceptable carrier is in the range of 80:20:0 to 89:9:2.

The Vitex negundo aqueous leaf extract powder is standardized for flavonoids and iridoid glycosides. The 0.7-3.0% iridoid glycosides can be selected from agnuside and negundoside or combination thereof. The flavonoids in Vitex negundo aqueous leaf extract powder will be present in an amount not less than 10% w/w.

The Zingiber officinale rhizome oleoresin is supercritical CO₂ extracted oleoresin standardized to at least one phytochemical selected from phenolic compounds. The phenolic compound can be gingerols. The gingerols in the oleoresin can be present not less than 30% w/w.

Another embodiment of the present disclosure discloses a process of preparation of a herbal composition comprising a) extracting of Vitex negundo leaf with a solvent followed by spraying and drying to obtain a Vitex negundo aqueous leaf extract powder; b) extracting oleoresin of Zingiber officinale rhizome by supercritical CO₂ extraction to get Zingiber officinale rhizome oleoresin; c) blending of Vitex negundo aqueous leaf extract powder of step (a) with supercritical CO₂ extracted Zingiber officinale rhizome oleoresin of step (b) to get a blend; d) drying the blend of step (c) with or without a pharmaceutical acceptable carrier to get free flowing powder followed by standardization to obtain a herbal composition standardized for flavonoids not less than 15%, iridoid glycosides not less than 0.4% can be selected from agnuside and negundoside or combination, and gingerols content not less than 2% wherein the ratio of Vitex negundo aqueous leaf extract powder to supercritical CO₂ Zingiber officinale rhizome oleoresin to pharmaceutical acceptable carrier is in the range of 80:20:0 to 89:9:2.

In one specific embodiment, the pharmaceutical acceptable carrier is silicon dioxide 0-2%.

In another embodiment, the present disclosure provides use of the herbal composition comprising Vitex negundo aqueous leaf extract powder; Zingiber officinale rhizome oleoresin for one or more conditions without limitation, to control acute or chronic musculoskeletal pain, physical-activity induced joint pain and related dysfunction.

The herbal composition comprising Vitex negundo aqueous leaf extract powder and Zingiber officinale rhizome oleoresin is effective for use in one or more of reducing acute or chronic musculoskeletal pain including osteoarthiris, rheumatoid arthritis and muscle pain, alleviating physical-activity induced joint pain such as mechanical loads on joints, reducing exercise-induced knee joint pain and chronic non-specific low back pain and related dysfunction.

Another embodiment of the present disclosure discloses a method for treating or preventing acute or chronic musculoskeletal pain, physical-activity induced joint pain and related dysfunction comprising administering an effective amount of the herbal composition to a subject in need of such treatment.

The inventors of the present invention surprisingly found that the herbal composition comprising Vitex negundo aqueous leaf extract powder and Zingiber officinale rhizome oleoresin helps in reduce joint pain by alleviating mechanical loading of the joints as assessed by change in TGF-β levels by 30% and more in 60-180 mins.

The inventors of the present invention found that the herbal composition comprising Vitex negundo aqueous leaf extract powder and Zingiber officinale rhizome oleoresin helps in reduce pain sensation by antagonising neurokinin-1 (Substance-P)

The present invention is a herbal composition comprising Vitex negundo aqueous leaf extract powder and Zingiber officinale rhizome with anti-inflammatory properties as demonstrated by decrease in IL-6, IL-4 and TNF-α levels.

The present invention is a herbal composition comprising Vitex negundo aqueous leaf extract powder and Zingiber officinale rhizome helps in cartilage regeneration as assessed by increase in Glial fibrillary acidic protein (GFAP) levels.

The preferable route of administration is oral administration.

The herbal composition the herbal composition comprising Vitex negundo aqueous leaf extract powder; Zingiber officinale rhizome oleoresin can be provided in a form selected from the group consisting of a solution, dispersion, suspension, powder, capsule, tablet, pill, time release capsule, time release tablet, and time release pill, preferably capsule, tablet and pill, gummies & chewie. The herbal composition is administered in the range of from about 0.001 mg per kg body weight per day to about 100 mg per kg body weight per day. The herbal composition is administered at least once daily. The subject is a mammalian subject.

The herbal composition comprising standardized extract of Vitex negundo leaves and Zingiber officinale rhizome oleoresin fills the gap of a safe and quick-acting pain-relieving product that can be used by any group of individuals without fear of the side effects of the popular pain-relieving synthetic drugs like NSAIDs.

While the foregoing describes various embodiments of the disclosure, other and further embodiments of the disclosure may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

### EXAMPLE

The disclosure will now be illustrated with the following example, which is intended to illustrate the working of disclosure as per one of the embodiments and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein. It is to be understood that this disclosure is not limited to particular methods, and experimental conditions described, as such methods and conditions may vary.

### Example 1

### Composition Containing Vitex Negundo Aqueous Leaf Extract and Zingiber Officinale Oleoresin

The Vitex negundo aqueous extract used in the herbal composition was obtained by water extraction process. Vitex negundo leaf were procured and authenticated using HPTLC ID method, were pulverised and extracted with 10 volume water at 90° C. for 2 hrs. The extraction cycles were repeated 5 times. Obtained water soluble extracts were mixed, concentrated, stripped and spray dried to get powdered Vitex negundo leaf aqueous extract standardized for flavonoids not less than 10%, iridoid glycosides is in the range of 0.7-3% can be selected from Agnuside and Negundoside or combination with herb to extract ratio 5:1.

The Zingiber officinalis rhizome extract of the present invention was obtained by super critical CO₂ extraction process. The Zingiber officinalis rhizomes were authenticated by HPTLC ID method, were extracted using supercritical CO₂ extraction method to get oleoresin standardized for total gingerols not less than 30% with herb to extract ratio was 35:1. Vitex negundo aqueous leaf extract) standardized for flavonoids not less than 10% and iridoid glycosides is in the range of 0.7-3.0% comprising of Agnuside and Negundoside or combination; Zingiber officinale super critical carbon dioxide extracted oleoresin) standardized for phenolic compounds specifically total Gingerols not less than 30%. The product is coded as E-PR-01.

### Example 2

Effect of the Composition Containing Vitex negundo Aqueous Leaf Extract and Zingiber officinale Oleoresin on Non-Specific Low Back Pain and Associated Conditions

Studies were conducted to evaluate the effectiveness of the composition comprising the Vitex negundo extract and the Zingiber officinale as obtained in Example 1 in alleviating non-specific low back pain, reducing disability and increasing work productivity as measured by changes in baseline versus end point scores in: a Pain VAS; Roland-Morris Low Back Pain and Disability Questionnaire (RMQ) score and Work Productivity and Activity Impairment Questionnaire. The composition E-PR-01 comprising the Vitex negundo extract and the Zingiber officinale oleoresin (178 mg+22 mg) and the placebo capsules containing the same amount of microcrystalline cellulose were used. All the participants were administered the assigned study product at a dose of one capsule orally after a baseline exercise session on Day 1, followed by one capsule daily after breakfast and before dinner for five days. All the participants were randomized to the alternate arm with a washout period of 5±2 days.

The composition E-PR-01 comprising the Vitex negundo extract and the Zingiber officinale oleoresin demonstrate quick onset of action and provide clinically meaningful relief within 2.4 hours of a single serving of 200 mg. The composition E-PR-01 was found to be effective in providing statistically significant treatment response with mean pain reduction as elucidated below:

### (a). Effect on Low Back Pain Intensity

Both E-PR-01 and placebo were able to reduce the pain intensity to a statistically significant extent by day 7; however, the magnitude of reduction observed in the E-PR-01 group was more than 3 times (16.23% vs. 5.06%) of that noted in the placebo group, and this difference between the two groups was highly significant (p<0.0012).

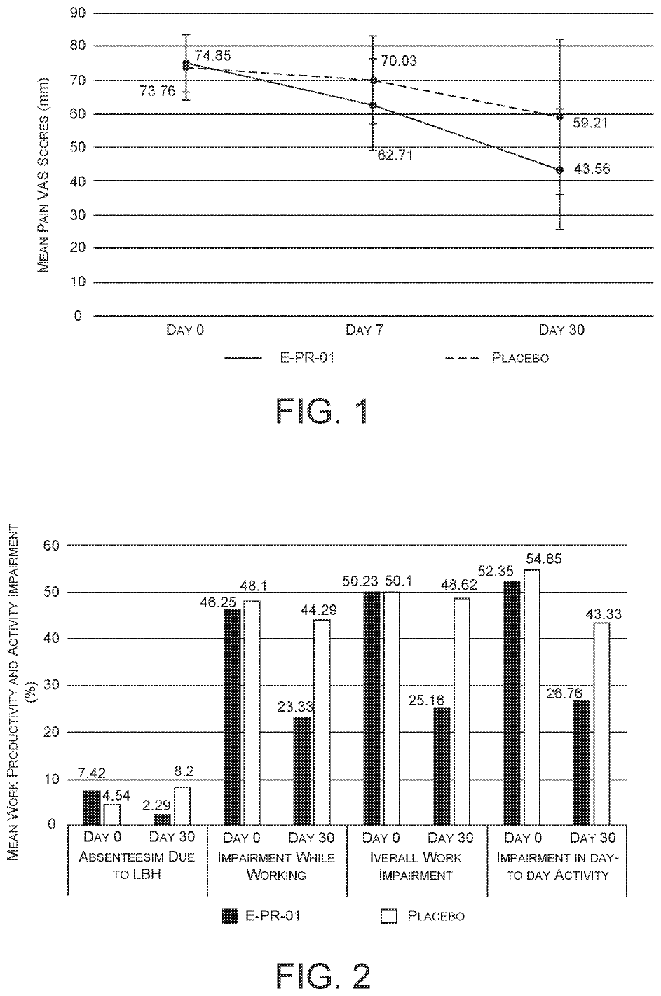

On day 30, the improvement in pain VAS score in the E-PR-01 group increased significantly from 16.23% to 41.8%, which was twice the improvement seen in the placebo group (19.73%). This result shows a highly significant effect of the E-PR-01 group on pain intensity compared to placebo (p<0.0002) (Table 1 & FIG. 1).

TABLE 1
Summary of fingertip to floor test scores (cm)

| Outcome | | E-PR-01 (N = 34) | | Placebo (N = 33) | | |
|---|---|---|---|---|---|---|
| variable | Visit | Mean (SD) | 95% C.I. | Mean (SD) | 95% C.I. | p-value^{a} |
| Fingertip-to- | Day 0 | 14.22 (7.11) | 11.74, 16.70 | 14.08 (6.22) | 11.88, 16.29 | 0.9340 |
| Floor Test | Day 30 | 8.62 (5.69) | 6.64, 10.60 | 11.71 (6.49) | 9.40, 14.01 | |
| | Change at | −5.60 (5.60) | −7.55, −3.64 | −2.38 (3.95) | −3.78, −0.97 | 0.0036 |
| | day 30 | | | | | |
| | p-value^{b} | <0.0001 | | 0.0016 | | |
| Pain VAS | Day 0 | 74.85 (8.53) | 71.88, 77.83 | 73.76 (9.66) | 70.33, 77.18 | 0.6242 |
| (mm) | Day 7 | 62.71 (13.58) | 57.97, 67.45 | 70.03 (12.96) | 65.43, 74.63 | |
| | Day 30 | 43.56 (17.81) | 37.34, 49.77 | 59.21 (23.07) | (51.03, 67.39) | |
| | Change on | −12.15 (11.61) | −16.20, −8.10 | −3.73 (8.00) | −6.56, −0.89 | 0.0012 |
| | day 7 | | | | | |
| | p-value^{b} | <0.0001 | | 0.0116 | | |
| | Change at | −31.29 (15.29) | −36.63, −25.96 | −14.55 (19.52) | −21.47, −7.62 | 0.0002 |
| | day 30 | | | | | |

| | | |
|---|---|---|
| p-value^{b} | <0.0001 | 0.0002 |

### (b) Effect on Work Productivity

### (i) Absenteeism Due to Low Back Pain

Out of 67 participants analyzed only, 45 participants were professionally engaged, 24 in the E-PR-01 group, and 21 in the placebo group were evaluated for absenteeism from professional engagement. The total working hours in the E-PR-01 and the placebo were almost similar at baseline (mean (SD): 38.52 (14.50) vs. 39.48 (11.89); p=0.8118) and remained the same at the end of the study (mean (SD): 36.92 (10.45) vs. 38.57 (13.38); p=0.6375) also.

On day 30, there was a reduction in the mean percentage of work time missed due to health in the E-PR-01 group; however, the magnitude of change from baseline was not statistically significant (p>0.05) (Table 2 & FIG. 2).

(ii) Impairment while Working Due to Low Back Pain

The mean percentage of impairment while working due to low back pain was assessed for the same number of participants as previous and was comparable for both the groups with no statistically significant intergroup difference (p=0.8075). A statistically significant reduction in the percentage from baseline values was observed in the E-PR-01 group, while no significant reduction was noticed in the placebo group. The percentage reduction in the E-PR-01 group was statistically significant (p<0.05) compared to the placebo group (Table 2 & FIG. 2).

(iii) Overall Work Impairment Due to Low Back Pain

The improvement in the percentage of overall work impairment during the professional engagement after the 30-day administration of E-PR-01 was statistically significant compared to the baseline as well as the placebo group (p<0.0001). However, the placebo group almost showed no improvement in the overall work impairment. (Table 2 & FIG. 2)

### (iv) Impairment in Day-to-Day Activities

Percentage of activity impairment in regular daily activity impairment other than professional engagement was assessed in 67 participants. After 30 days of intervention, there was a statistically significant reduction in both groups (p<0.05). However, the magnitude of change in percentage in the E-PR-01 group was more than twice the change observed in the placebo, and this intergroup difference was statistically significant (p=0.0015) (Table 2 & FIG. 2).

TABLE 2
Summary of work productivity and activity impairment questionnaire scores.

| | | E-PR-01 (N = 34) | | Placebo (N = 33) | | |
|---|---|---|---|---|---|---|
| Parameter | Change | Mean (SD) | 95% C.I. | Mean (SD) | 95% C.I. | p-value^{a} |
| Absenteeism due | N | 24 | | 20^{c} | | |
| to low back pain | Change at | −5.13 (12.97) | −10.60, 0.35 | 3.65 (20.08) | −5.75, 13.04 | 0.1452 |
| (%) | day 30 | | | | | |
| | p-value^{b} | 0.0651 | | 0.4267 | | |
| Impairment | N | 24 | | 21 | | |
| while working | Change at | −22.92 (17.06) | −30.12, −15.71 | −3.81 (18.57) | 12.26, 4.64 | <0.0001 |
| due to low back | day 30 | | | | | |
| pain | p-value^{b} | <0.0001 | | 0.3583 | | |
| (%) | | | | | | |
| Overall work | N | 24 | | 20^{c} | | |
| impairment due | Change at | −25.06 (20.26) | −33.62, −16.51 | −0.42 (19.65) | −9.62, 8.78 | <0.0001 |
| to low back pain | day 30 | | | | | |
| (%) | p-value^{b} | <0.0001 | | 0.9251 | | |
| Impairment in | N | 34 | | 33 | | |
| day-to-day | Change at | −25.59 (17.95) | −31.85, −19.32 | −11.52 (21.95) | −19.30, −3.73 | 0.0015 |
| activities | day 30 | | | | | |

| | | | |
|---|---|---|---|
| (%) | p-value^{b} | <0.0001 | 0.0050 |

^{a}p-value was calculated using ANCOVA with treatment as factor and baseline as covariate vs. placebo.
^{b}p-value was calculated using paired t-test.
^{c}One observation was invalid, as the participant was on vacation on Day 30.

### (c) Effect on Sleep Quality

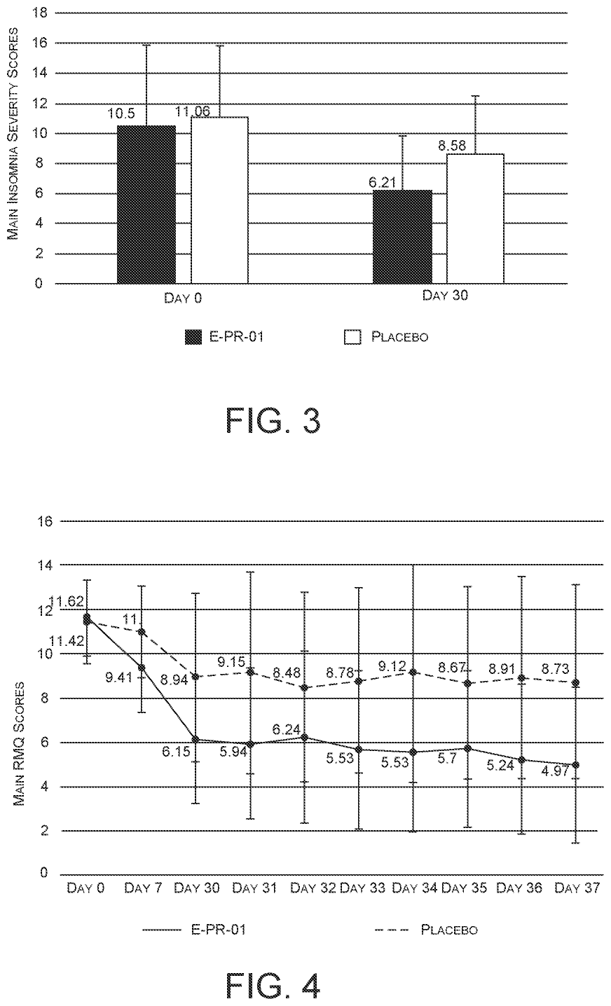

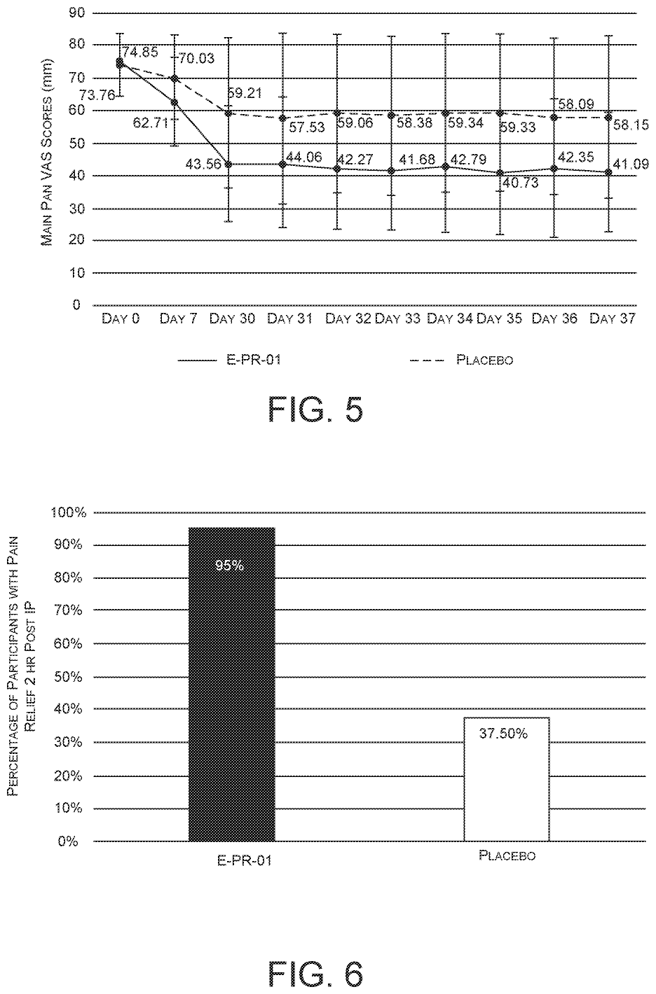

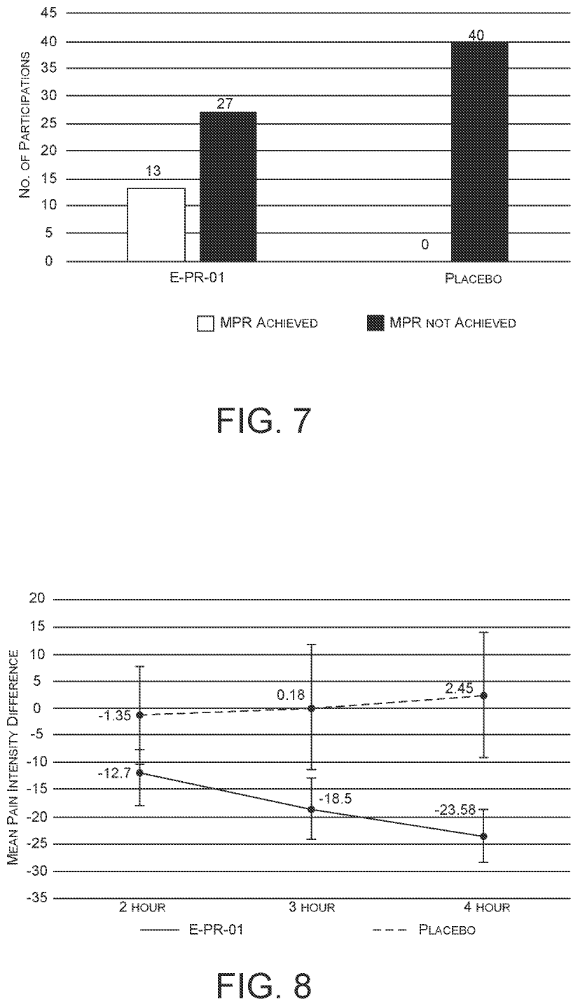

The baseline mean insomnia severity in both the study groups was at the subthreshold level and comparable (p=0.6526) between the groups. After 30 days of intervention, a statistically significant reduction in mean insomnia severity scores was observed in both study groups. However, the magnitude of change was significantly higher in the E-PR-01 group compared to the placebo (−4.29 vs. −2.48). The difference in the change observed was statistically significant when compared between the two groups. This result shows that the 30-day administration of E-PR-01 was able to decrease the mean severity of insomnia from subthreshold to clinically insignificant levels, while in the placebo group, it remained at the same level (Table 3& FIG. 3).

TABLE 3
Summary of the insomnia severity index

| | E-PR-01 (N = 34) | | Placebo (N = 33) | | |
|---|---|---|---|---|---|
| Visit | Mean (SD) | 95% C.I. | Mean (SD) | 95% C.I. | p-value^{a} |
| Day 0 | 10.50 (5.36) | 8.63, 12.37 | 11.06 (4.76) | 9.37, 12.75 | 0.6526 |
| Day 30 | 6.21 (3.62) | 4.94, 7.47 | 8.58 (3.91) | 7.19, 9.96 | |
| Change at day 30 | −4.29 (4.12) | −5.73, −2.86 | −2.48 (4.06) | −3.92, −1.05 | 0.0056 |

| | | |
|---|---|---|
| p-value^{b} | <0.0001 | 0.0013 |

^{a}p-value was calculated using ANCOVA with treatment as factor and baseline as covariate vs. placebo.
^{b}p-value was calculated using paired t-test.

### (d) Sustained Effect of the Product

The sustained effect of the product was measured over a follow-up period of 7 days after the cessation of the intervention by recording daily pain intensity on pain VAS and functional activity scores on RMQ. During this period, the participants were instructed to abstain from using any pain-relieving oral medications or topical applications. Both the groups showed a statistically significant sustained effect on the pain VAS scores and the functional activity till day 7. However, in the E-PR-01 group, the sustained effect was almost twice the magnitude noticed in the placebo group for both the pain intensity and the functional activity scores, and the difference between the two groups was also statistically significant (Table 4, 5 & FIGS. 4 & 5).

TABLE 4
Sustained effect of IP on RMQ scores during the 7-day follow-up period.

| | E-PR-01 | | Placebo | | |
|---|---|---|---|---|---|
| Visit | Mean (SD) | 95% CI | Mean (SD) | 95% CI | p-value^{a} |
| Day 30 | N = 34 | | N = 33 | | |
| | 6.15 (2.89) | 5.14-7.16 | 8.94 (3.79) | 7.60-10.28 | |
| Day 37 | N = 33 | | N = 33 | | |
| | 4.97 (3.54) | 3.71-6.22 | 8.73 (4.38) | 7.17-10.28 | |
| Change | N = 33 | | N = 33 | | <0.0001 |
| | −6.61 (3.57) | −7.87-−5.34 | −2.70 (3.84) | −4.06-−1.33) | |

| | | |
|---|---|---|
| p-value^{b} | <0.0001 | 0.0003 |

^{a}p-value was calculated using ANCOVA with treatment as factor and baseline as covariate vs. placebo.
^{b}p-value was calculated using paired t-test.

TABLE 5
Sustained effect of IP on pain VAS scores during the 7-day follow-up period

| | E-PR-01 | | Placebo | | |
|---|---|---|---|---|---|
| Visit | Mean (SD) | 95% CI | Mean (SD) | 95% CI | p-value^{a} |
| Day 30 | N = 34 | | N = 33 | | |
| | 43.56 (17.81) | 37.34, 49.77 | 59.21 (23.07) | (51.03, 67.39) | |
| Day 37 | N = 33 | | N = 33 | | |
| | 41.09 (18.38) | 34.47-47.72 | 58.15 (24.91) | 49.32-66.99 | |
| Change | N = 33 | | N = 33 | | <0.0005 |
| | −33.91 (17.45) | −40.09-−27.72 | −15.61 (22.14) | −23.46-−7.75 | |

| | | |
|---|---|---|
| p-value^{b} | <0.0001 | 0.0003 |

^{a}p-value was calculated using ANCOVA with treatment as factor and baseline as covariate vs. placebo.
^{b}p-value was calculated using paired t-test.

### (e) Safety Outcomes

No significant change was observed in the vitals of the participants in both groups during the 30-day study period. (Table 6)

TABLE 6
Change for Vital Signs - mITT population

| | E-PR-01 (N = 35) | | Placebo (N = 33) | | |
|---|---|---|---|---|---|
| Parameters | Mean (SD) | 95% C.I. | Mean (SD) | 195% C.I. | value |
| SBP (mmHg) | 1.31 (6.56) | −0.94, 3.57 | 0.36 (7.27) | −2.21, 2.94 | 0.5729 |
| p-value^{b} | 0.2442 | | 0.7757 | | |
| DBP (mmHg) | 1.20 (6.10) | −0.90, 3.30 | 2.06 (4.96) | 0.30, 3.82 | 0.5271 |
| p-value^{b} | 0.2527 | | 0.0231 | | |
| Pulse Rate | 0.49 (8.94) | −2.59, 3.56 | −1.00 (4.83) | −2.71, 0.71 | 0.3942 |
| (Beats/min.) | | | | | |

| | | |
|---|---|---|
| p-value^{b} | 0.7499 | 0.2429 |

^{a}p-value was calculated using ANCOVA with treatment as factor and baseline as covariate vs. placebo.
^{b}p-value was calculated using paired t-test.

Two adverse events (dry cough, n=1; fever and body ache, n=1) were reported in the E-PR-01 group, which were unrelated to the product, and both the adverse events were resolved without any consequences.

Based on the results of the present study and their comparisons with previous similar studies, we can say that both Vitex negundo and Zingiber officinale oleoresin worked synergistically, producing a remarkable effect in controlling the pain and functional disability due to low back pain without a specific etiology, a prevalent condition in today's young as well as middle-aged generation who are having prolonged sitting hours at work or home. Additionally, it improved the overall quality of life by improving their work productivity and sleep quality.

The composition was found to be effective to alleviate non-specific low back pain, reducing disability and increasing work productivity as measured by changes in baseline versus end point scores in: a Pain VAS; Roland-Morris Low Back Pain and Disability Questionnaire (RMQ) score and Work Productivity and Activity Impairment Questionnaire. The composition comprising the Vitex negundo extract and the Zingiber officinale oleoresin demonstrate quick onset of action and provide clinically meaningful relief within 2.4 hours of a single serving of 200 mg.

### Example 3

Effect of the Composition Containing Vitex negundo Aqueous Leaf Extract and Zingiber officinale Oleoresin on Exercise-Induced Acute Knee Joint Pain

E-PR-01 capsule containing 200 mg of extracts of Vitex negundo and Zingiber officinale, and the placebo capsules containing the same amount of microcrystalline cellulose were used in the studies. All the participants were administered the assigned study product at a dose of one capsule orally after a baseline exercise session on Day 1, followed by one capsule daily after breakfast and before dinner for five days. All the participants were randomized to the alternate arm with a washout period of 5±2 days.

### (a) Time to Meaningful Pain Relief

The mean time to achieve meaningful analgesia in the E-PR-01 group was 3.38 hours. 95% of participants in this group experienced perceptible pain relief at 2 hours after a single dose of IP administration; on the other hand, in the placebo group, no participant could achieve MPR till 4 hours; however, 37.5% of the participants receiving placebo, experienced perceptible pain relief at 2 hours (Table 7 and FIGS. 6 & 7). The difference for the number and percentage of participants achieving MPR was found to be statistically significant between the groups (p<0.001).

TABLE 7
Time to achieve MPR

| | E-PR-01 | Placebo |
|---|---|---|
| Time to achieve MPR | (n = 40) | (n = 40) |
| Mean (hours) | 3.38 | — |
| 95% CI of Mean | (2.92, 3.85) | — |
| SD | 0.77 | — |
| Minimum (hours) | 2.00 | — |
| | E-PR-01 | Placebo |
| Time to Achieve MPR | (n = 40) | (n = 40) |
| Maximum (hours) | 4.00 | — |

### (b) Pain Intensity Difference

The post-exertion absolute and percentage pain intensity difference on day 1, 2-, 3- and 4-hour after single-dose administration of E-PR-01 was statistically significant compared to the placebo group (all p=<0.001). In the E-PR-01 group, there was a continuous reduction in post-exertion pain VAS scores from 19% at 2 hours to 35% at 4 hours post-IP administration from the baseline values, while in the placebo group, the pain VAS scores increased at 4 hours post-IP administration (4%) after an initial reduction at 2-hour post-IP administration (1.5%) (Table 8a & 8b and FIGS. 8 & 9).

TABLE 8A
Summary of pain VAS score

| | | E-PR-01 (n = 40) | | Placebo (n = 40) | | |
|---|---|---|---|---|---|---|
| Parameter | Visit (Day) | Mean (SD) | 95% CI | Mean (SD) | 95% CI | p-value |
| Pain VAS | Pre-exertion | 23.95 (5.95) | 22.05-25.85 | 23.5 (6.01) | 21.58-25.42 | — |
| Score (mm) | Post-exertion scores - Day 1 | | | | | |
| | Day 1 - 0 hour | 67.45 (7.36) | 65.10-69.80 | 66.68 (6.00) | 64.76-68.59 | 0.6073 |
| | Day 1 - 2 hours | 54.75 (10.25) | 51.47-58.03 | 65.33 (5.72) | 63.50-67.15 | — |
| | Day 1 - 3 hours | 48.95 (12.33) | 45.01-52.89 | 66.85 (5.55) | 65.08-68.62 | — |
| | Day 1 - 4 hours | 43.88 (12.43) | 39.90-47.85 | 69.13 (5.53) | 67.36-70.89 | — |
| | Day 5 | 34.20 (10.11) | 30.97-37.43 | 67.80 (6.01) | 65.88-69.72 | — |

TABLE 8B
Summary of PID and SPID

| | | E-PR-01 (n = 40) | | Placebo (n = 40) | | |
|---|---|---|---|---|---|---|
| Parameters | Visit (Day) | Mean (SD) | 95% CI | Mean (SD) | 95% CI | p-value |
| PID (mm) | Day 1 - 2 hours | −12.7 (9.08) | −15.60-−9.80 | −1.35 (5.15) | −3.00-0.30 | <0.0001 |
| | Day 1 - 3 hours | −18.5 (11.56) | −22.20-−14.80 | 0.18 (5.62) | −1.62-1.97 | <0.0001 |
| | Day 1 - 4 hours | −23.58 (11.49) | −27.25-−19.90 | 2.45 (4.77) | 0.92-3.98 | <0.0001 |
| SPID (mm) | Day 1 | −67.48 (39.62) | −80.15-−54.80 | −0.08 (16.98) | −5.51-5.36 | <0.0001 |

### (c) Time-Weighted Sum of Pain Intensity Difference

The time-weighted sum of pain intensity difference was significantly more in the E-PR-01 group compared to the placebo group (p<0.0001) after single-dose administration.

### (d) Responder Analysis

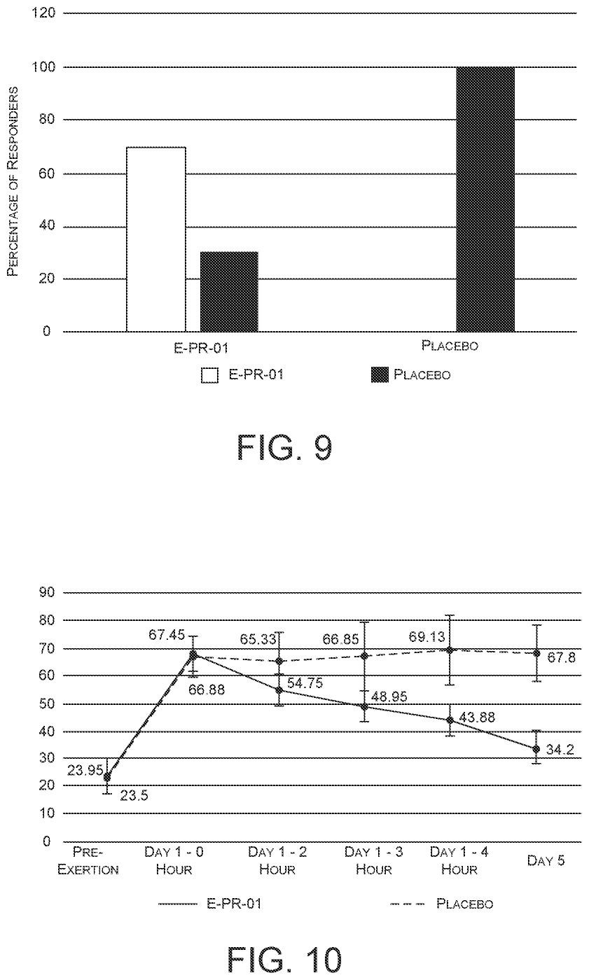

In the E-PR-01 group, the responders almost doubled with each passing hour on day 1 (post-exertion) 2 hours, 3 hours, 4 hours, and day 5 (post-exertion) (4 hours), respectively. In placebo, no responders were observed. On Day 5, there was a significant difference in the cumulative number of responders in both groups (p<0.0001). (Table 9 & FIG. 9)

TABLE 9
Cumulative number and percentage of responders
and non-responders on Day 5.

| | | E-PR-01 (n = 40) | Placebo (n = 40) | |
|---|---|---|---|---|
| Visit | Outcome | n (%) | | p-value |
| Day 5 | Responders | 28 (70.00%) | 0 (0.00%) | <0.0001b |
| | Non- | 12 (30.00%) | 40 100.00%) | |
| | responders | | | |

### (e) Pain VAS Score on Day 5

Pain VAS scores were statistically comparable between the two groups at baseline (p>0.05). On day 5 a significant reduction (50%) in mean VAS scores from the baseline scores was observed in the E-PR-01 group (p<0.001). In addition, this improvement in the pain scores in the E-PR-01 group was statistically significant (p<0.001) compared to the placebo group, which almost remained the same, as shown in Table 10 & FIG. 10.

TABLE 10
Summary of change in pain VAS scores from day 1 to day 5

| | E-PR-01 (n = 40) | | Placebo (n = 40) | | |
|---|---|---|---|---|---|
| Categories | Mean (SD) | 95% CI | Mean (SD) | 95% CI | p-value |
| Day 1 - | 67.45 (7.36) | 65.10-69.80 | 66.68 (6.00) | 64.76-68.59 | 0.6073 |
| 0 hour | | | | | |
| Day 5 | 34.20 (10.11) | 30.97-37.43 | 67.80 (6.01) | 65.88-69.72 | — |
| Change | −33.25 (11.09) | −36.80-−29.70 | 1.13 (4.43) | −0.29-2.54 | <0.0001 |

| | | |
|---|---|---|
| p-value^{b} | <0.0001 | 0.1165 |

### (f) Physical Efficiency

All subjects completed exercise sessions at 4-hour post-IP on day 1 and day 5 in both the study groups without any debilitating pain, defined as pain VAS score>90 mm).

### (g) Safety Outcomes

There was no significant change in the vitals of the participants, and no adverse event was reported in both the study groups during the entire study duration.

Overall, E-PR-01 significantly improved the exercise-induced acute knee joint pain at 2 hours which continued till 4 hours after a single dose of 200 mg. This pain relief was considered meaningful by the participants. It was found to be safer than the conventional treatments compared to the results obtained in similar studies.

### Example 4

Comparative Study to Investigate the Effect of the Composition Containing Vitex negundo Aqueous Leaf Extract and Zingiber officinale Oleoresin of the Present Invention in Comparison with Different Individual Ingredients and Combination in Relieving Joint Pain Associated with Physical Activity

A comparative study was conducted to investigate the effect of the composition of the present invention E-PR-01 obtained in Examples 1 in comparison with leaf extract of Pluchea lanceolata individually and various combinations of Zingiber officinale, Vitex negundo, Pluchea lanceolata, Alpinia galanga, Boswellia serrata, Psidium guajava, and Kaempferia parviflora extracts as per Table 11, for relieving joint pain associated with physical activity.

The study was conducted in three consecutive phases—Phase I, Phase II, and Phase III with a different group of Investigational products (IPs), as described in Table 11.

TABLE 11
Details of Investigational products.

| S. | Research | | Dose and |
|---|---|---|---|
| No. | code | Composition | regimen |
| 1 | Placebo | Microcrystalline cellulose (500 mg) | One |
| 2 | PL-L | Pluchea lanceolata leaf (300 mg) | capsule |
| 3 | PL-H | Pluchea lanceolata leaf (600 mg) | only |
| 4 | PL-E | Pluchea lanceolata leaf (300 mg) + | |
| | | Alpinia galanga rhizome (300 mg) | |
| 5 | BS-ZP | Boswellia serrata (220 mg) + | |
| | | Zingiber officinale rhizome (80 mg) + | |
| | | Pluchea lanceolata leaf (100 mg) | |
| 6 | PL-Z | Zingiber officinale rhizome (100 mg) + | |
| | | Pluchea lanceolata leaf (150 mg) | |
| 7 | ZP | Zingiber officinale rhizome (100 mg) + | |
| | | Psidium guajava leaf (300 mg) | |
| 8 | ZV | Vitex negundo leaf (178 mg) + | |
| | | Zingiber officinale rhizome (22 mg) | |
| 9 | KV | Kaempferia parviflora rhizome (100 mg) + | |
| | | Vitex negundo leaf (300 mg) | |

### (a) Time Taken to Achieve MPR

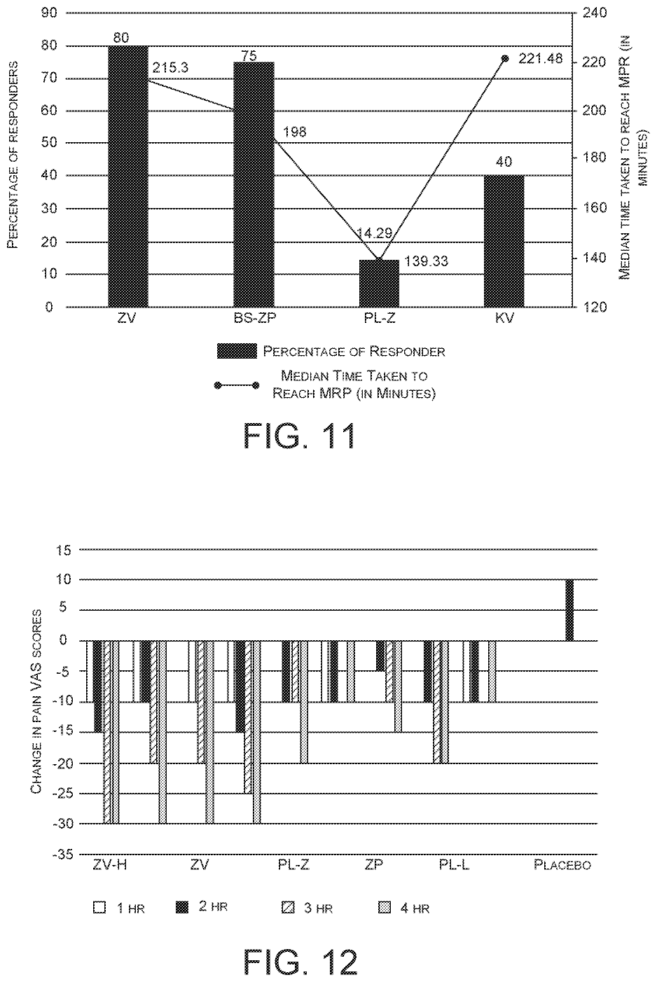

MPR, as defined by a reduction of 30 mm on the 100-mm pain VAS scale, was successfully achieved in more than one participant in five out of eleven of the IP groups, as shown in Table 12. The highest number of participants (80%) in the ZV group achieved MPR, which was followed by BS-ZP (75%). On the other hand, in KV and PL-Z groups, only 40% and 14.29% of participants experienced MPR, respectively. All the participants in ZP, PL-E, PL-L, PL-H, and Placebo groups failed to achieve MPR (Tables (12 and 13) and FIG. 11).

TABLE 12
Number of participants with MPR

| | | |
|---|---|---|
| MPR achieved | MPR not achieved | Total |

| IP | N | % | N | % | n |
|---|---|---|---|---|---|
| Placebo | 0 | 0 | 5 | 100 | 5 |
| PL-L | 0 | 0 | 5 | 100 | 5 |
| PL-H | 0 | 0 | 3 | 100 | 3 |
| PL-E | 0 | 0 | 3 | 100 | 3 |
| BS-ZP | 3 | 75 | 1 | 25 | 4 |
| PL-Z | 1 | 14.29 | 6 | 85.71 | 7 |
| ZP | 0 | 0 | 4 | 100 | 4 |
| ZV | 4 | 80 | 1 | 20 | 5 |
| KV | 2 | 40 | 3 | 60 | 5 |

TABLE 13
Time taken to achieve MPR (minutes)

| IP | N | Mean | SD | Min. | p25 | Median | p75 | Max. |
|---|---|---|---|---|---|---|---|---|
| Placebo | 5 | MPR Not Achieved | | | | | | |
| PL-L | 5 | | | | | | | |
| PL-H | 3 | | | | | | | |
| PL-E | 3 | | | | | | | |
| BS-ZP | 4 | 208.72 | 44.89 | 170.2 | 170.17 | 198 | 258 | 258 |
| PL-Z | 7 | 139.33 | . | 139.3 | 139.33 | 139.33 | 139.33 | 139 |
| ZP | 4 | MPR Not Achieved | | | | | | |
| ZV | 5 | 201.52 | 31.24 | 155.2 | 182.79 | 215.3 | 220.24 | 220 |
| KV | 5 | 221.48 | 22.43 | 205.6 | 205.62 | 221.48 | 237.33 | 237 |

### (b) Pain Intensity

Table 13 & FIG. 12 shows the pre and post-exertion pain VAS scores for all the groups at different time points. Irrespective of the number of participants achieving MPR, ZV group followed by and BS-ZP demonstrated a clinically meaningful decrease of at least 30 mm in pain VAS scores post-exertion, as compared to the baseline (FIG. 12). However, the 75th percentile of the data (p75) showed that in the ZV group, the pain VAS score was lower than 40 in 75% of participants, while in the other groups, this value (p75) was more than 40.

TABLE 13
Pre and post-exertion VAS scores (mm)

| Time- | Pre-exertion | | | | | Post-exertion | | | | |
|---|---|---|---|---|---|---|---|---|---|---|
| points | Mean | SD | Median | p25 | p75 | Mean | SD | Median | p25 | p75 |

Placebo (n = 5)

| | | | | | | | | | | |
|---|---|---|---|---|---|---|---|---|---|---|
| 0 hr | 20 | 7.07 | 20 | 20 | 20 | 70 | 7.07 | 70 | 70 | 70 |
| 1 hr | 52 | 19.24 | 50 | 40 | 60 | 72 | 8.37 | 70 | 70 | 80 |
| 2 hr | 54 | 15.17 | 50 | 40 | 70 | 76 | 5.48 | 80 | 70 | 80 |
| 3 hr | 56 | 13.42 | 50 | 50 | 70 | 74 | 8.94 | 80 | 70 | 80 |
| 4 hr | 52 | 17.89 | 50 | 40 | 70 | 74 | 8.94 | 80 | 70 | 80 |

PL-L (n = 5)

| | | | | | | | | | | |
|---|---|---|---|---|---|---|---|---|---|---|
| 0 hr | 24 | 5.48 | 20 | 20 | 30 | 72 | 4.47 | 70 | 70 | 70 |
| 1 hr | 50 | 10 | 50 | 40 | 60 | 64 | 5.48 | 60 | 60 | 70 |
| 2 hr | 50 | 10 | 50 | 40 | 60 | 66 | 8.94 | 60 | 60 | 70 |
| 3 hr | 50 | 15.81 | 50 | 40 | 60 | 68 | 14.83 | 70 | 60 | 70 |
| 4 hr | 48 | 13.04 | 50 | 40 | 60 | 64 | 16.73 | 60 | 50 | 70 |

PL-H (n = 3)

| | | | | | | | | | | |
|---|---|---|---|---|---|---|---|---|---|---|
| 0 hr | 23.33 | 5.77 | 20 | 20 | 30 | 66.67 | 5.77 | 70 | 60 | 70 |
| 1 hr | 50 | 17.32 | 60 | 30 | 60 | 73.33 | 5.77 | 70 | 70 | 80 |
| 2 hr | 46.67 | 23.09 | 60 | 20 | 60 | 70 | 10 | 70 | 60 | 80 |
| 3 hr | 50 | 17.32 | 60 | 30 | 60 | 70 | 10 | 70 | 60 | 80 |
| 4 hr | 46.67 | 15.28 | 50 | 30 | 60 | 63.33 | 5.77 | 60 | 60 | 70 |

PL-E (n = 3)

| | | | | | | | | | | |
|---|---|---|---|---|---|---|---|---|---|---|
| 0 hr | 26.67 | 5.77 | 30 | 20 | 30 | 60 | 0 | 60 | 60 | 60 |
| 1 hr | 40 | 10 | 40 | 30 | 50 | 60 | 0 | 60 | 60 | 60 |
| 2 hr | 40 | 10 | 40 | 30 | 50 | 56.67 | 11.55 | 50 | 50 | 70 |
| 3 hr | 33.33 | 5.77 | 30 | 30 | 40 | 56.67 | 11.55 | 50 | 50 | 70 |
| 4 hr | 33.33 | 5.77 | 30 | 30 | 40 | 50 | 10 | 50 | 40 | 60 |

BS-ZP(n = 4)

| | | | | | | | | | | |
|---|---|---|---|---|---|---|---|---|---|---|
| 0 hr | 22.5 | 5 | 20 | 20 | 25 | 65 | 5.77 | 65 | 60 | 70 |
| 1 hr | 32.5 | 17.08 | 35 | 20 | 45 | 57.5 | 5 | 60 | 55 | 60 |
| 2 hr | 30 | 14.14 | 35 | 20 | 40 | 55 | 10 | 50 | 50 | 60 |
| 3 hr | 27.5 | 12.58 | 30 | 20 | 35 | 47.5 | 15 | 40 | 40 | 55 |
| 4 hr | 22.5 | 9.57 | 25 | 15 | 30 | 42.5 | 12.58 | 40 | 35 | 50 |

PL-Z (n = 7)

| | | | | | | | | | | |
|---|---|---|---|---|---|---|---|---|---|---|
| 0 hr | 24.29 | 5.35 | 20 | 20 | 30 | 64.29 | 5.35 | 60 | 60 | 70 |
| 1 hr | 28.57 | 10.69 | 30 | 20 | 30 | 61.43 | 12.15 | 70 | 50 | 70 |
| 2 hr | 30 | 16.33 | 20 | 20 | 50 | 55.71 | 13.97 | 60 | 50 | 70 |
| 3 hr | 28.57 | 14.64 | 20 | 20 | 40 | 55.71 | 17.18 | 60 | 40 | 70 |
| 4 hr | 28.57 | 14.64 | 20 | 20 | 40 | 51.43 | 15.74 | 50 | 40 | 70 |

ZP (n = 4)

| | | | | | | | | | | |
|---|---|---|---|---|---|---|---|---|---|---|
| 0 hr | 22.5 | 5 | 20 | 20 | 25 | 62.5 | 5 | 60 | 60 | 65 |
| 1 hr | 30 | 11.55 | 30 | 20 | 40 | 62.5 | 5 | 60 | 60 | 65 |
| 2 hr | 27.5 | 9.57 | 25 | 20 | 35 | 60 | 11.55 | 60 | 50 | 70 |
| 3 hr | 25 | 5.77 | 25 | 20 | 30 | 55 | 5.77 | 55 | 50 | 60 |
| 4 hr | 20 | 8.16 | 20 | 15 | 25 | 50 | 8.16 | 50 | 45 | 55 |

ZV (n = 5)

| | | | | | | | | | | |
|---|---|---|---|---|---|---|---|---|---|---|
| 0 hr | 28 | 4.47 | 30 | 30 | 30 | 64 | 5.48 | 60 | 60 | 70 |
| 1 hr | 34 | 11.4 | 30 | 30 | 40 | 60 | 0 | 60 | 60 | 60 |
| 2 hr | 32 | 13.04 | 30 | 20 | 40 | 52 | 4.47 | 50 | 50 | 50 |
| 3 hr | 28 | 16.43 | 20 | 20 | 40 | 46 | 8.94 | 40 | 40 | 50 |
| 4 hr | 28 | 16.43 | 20 | 20 | 40 | 40 | 12.25 | 40 | 30 | 40 |

KV (n = 5)

| | | | | | | | | | | |
|---|---|---|---|---|---|---|---|---|---|---|
| 0 hr | 26 | 5.48 | 30 | 20 | 30 | 62 | 4.47 | 60 | 60 | 60 |
| 1 hr | 34 | 11.4 | 30 | 30 | 40 | 58 | 8.37 | 60 | 50 | 60 |
| 2 hr | 34 | 11.4 | 30 | 30 | 40 | 52 | 13.04 | 50 | 40 | 60 |
| 3 hr | 32 | 8.37 | 30 | 30 | 40 | 50 | 14.14 | 40 | 40 | 60 |
| 4 hr | 28 | 10.95 | 30 | 30 | 30 | 42 | 13.04 | 40 | 30 | 50 |

### (c) Time-Weighted Sum of Pain Intensity Difference

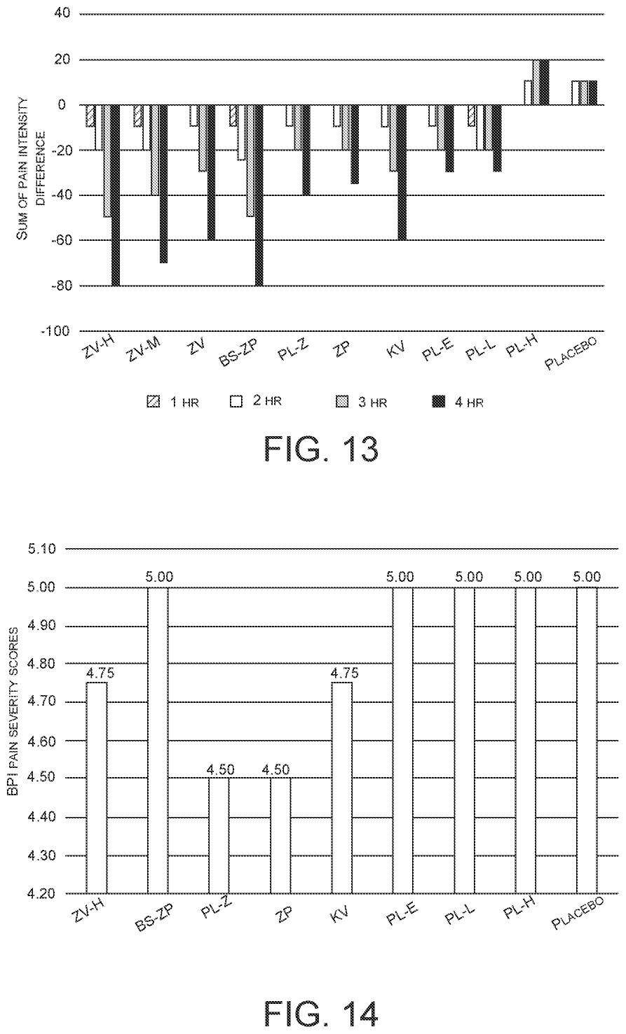

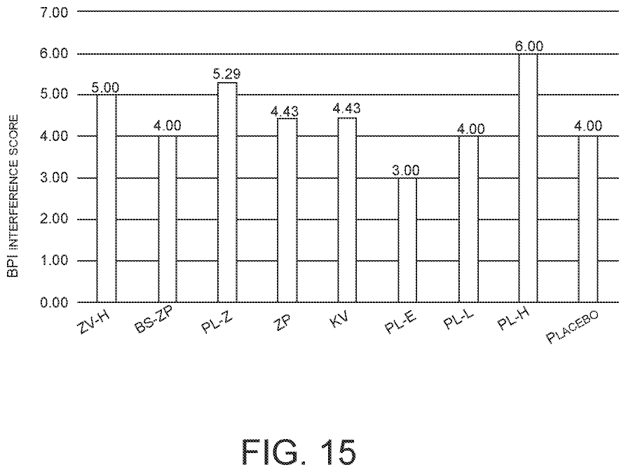

The time-weighted sum of pain intensity difference at 4-hour post-IP administration was observed to be reduced to a maximum level (80 mm) from baseline in the group followed BS-ZP, and KV group. The remaining groups, i.e., PL-Z, PL-E, PL-L. ZP also showed a reduction in the SPID in descending order but less than the above groups. On the contrary, Placebo and PL-H groups showed increased SPID by 20 and 10 mm, respectively, from baseline (Table 14 and FIG. 13).

TABLE 14
Time-weighted sum of pain intensity difference (mm)
SPID on Pain VAS

| | | | | | |
|---|---|---|---|---|---|
| Time-points | Mean | SD | Median | p25 | p75 |

Placebo (n = 5)

| | | | | | |
|---|---|---|---|---|---|
| 1 hr | 2 | 4.47 | 0 | 0 | 0 |
| 2 hr | 8 | 8.37 | 10 | 0 | 10 |
| 3 hr | 12 | 13.04 | 10 | 0 | 20 |
| 4 hr | 16 | 18.17 | 10 | 0 | 30 |

PL-L (n = 5)

| | | | | | |
|---|---|---|---|---|---|
| 1 hr | −8 | 4.47 | −10 | −10 | −10 |
| 2 hr | −14 | 8.94 | −20 | −20 | −10 |
| 3 hr | −18 | 17.89 | −20 | −30 | 0 |
| 4 hr | −26 | 30.5 | −30 | −50 | 0 |

PL-H (n = 3)

| | | | | | |
|---|---|---|---|---|---|
| 1 hr | 6.67 | 11.55 | 0 | 0 | 20 |
| 2 hr | 10 | 10 | 10 | 0 | 20 |
| 3 hr | 13.33 | 11.55 | 20 | 0 | 20 |
| 4 hr | 10 | 17.32 | 20 | −10 | 20 |

PL-E (n = 3)

| | | | | | |
|---|---|---|---|---|---|
| 1 hr | 0 | 0 | 0 | 0 | 0 |
| 2 hr | −3.33 | 11.55 | −10 | −10 | 10 |
| 3 hr | −6.67 | 23.09 | −20 | −20 | 20 |
| 4 hr | −16.67 | 32.15 | −30 | −40 | 20 |

BS-ZP(n = 4)

| | | | | | |
|---|---|---|---|---|---|
| 1 hr | −7.5 | 5 | −10 | −10 | −5 |
| 2 hr | −17.5 | 18.93 | −25 | −30 | −5 |
| 3 hr | −35 | 37.86 | −50 | −60 | −10 |
| 4 hr | −57.5 | 52.52 | −80 | −90 | −25 |

PL-Z (n = 7)

| | | | | | |
|---|---|---|---|---|---|
| 1 hr | −2.86 | 9.51 | 0 | −10 | 0 |
| 2 hr | −11.43 | 21.16 | −10 | −20 | 0 |
| 3 hr | −20 | 36.51 | −20 | −40 | 0 |
| 4 hr | −32.86 | 49.57 | −40 | −60 | 0 |

ZP (n = 4)

| | | | | | |
|---|---|---|---|---|---|
| 1 hr | 4 | 0 | 0 | −5 | 5 |
| 2 hr | 4 | −2.5 | −10 | −10 | 5 |
| 3 hr | 4 | −10 | −20 | −20 | 0 |
| 4 hr | 4 | −22.5 | −35 | −40 | −5 |

ZV (n = 5)

| | | | | | |
|---|---|---|---|---|---|
| 1 hr | −4 | 5.48 | 0 | −10 | 0 |
| 2 hr | −16 | 13.42 | −10 | −30 | −10 |
| 3 hr | −34 | 23.02 | −30 | −50 | −30 |
| 4 hr | −58 | 34.93 | −60 | −80 | −60 |

KV (n = 5)

| | | | | | |
|---|---|---|---|---|---|
| 1 hr | −4 | 5.48 | 0 | −10 | 0 |
| 2 hr | −14 | 15.17 | −10 | −30 | 0 |
| 3 hr | −26 | 25.1 | −30 | −50 | 0 |
| 4 hr | −46 | 33.62 | −60 | −70 | −10 |

### (d) Brief Pain Inventory

The median pain severity the following morning was almost similar across the groups. It ranged from 4.50 to 5.00. Only PL-L. ZV, and PL-Z groups showed scores lower than the placebo. Overall, the score ranged from mild to moderate (2.43-6.00) (Table 15 and FIGS. 14 & 15).

TABLE 15
Brief pain inventory scores

| | | | | | | | | |
|---|---|---|---|---|---|---|---|---|
| IP | N | Mean | SD | Median | p25 | p75 | Min. | Max. |

Pain severity score

| | | | | | | | | |
|---|---|---|---|---|---|---|---|---|
| Placebo | 5 | 5 | 0 | 5 | 5 | 5 | 5 | 5 |
| PL-L | 5 | 5.4 | 0.55 | 5 | 5 | 6 | 5 | 6 |
| PL-H | 3 | 5 | 0 | 5 | 5 | 5 | 5 | 5 |
| PL-E | 3 | 5 | 0 | 5 | 5 | 5 | 5 | 5 |
| BS-ZP | 4 | 4.75 | 0.5 | 5 | 4.5 | 5 | 4 | 5 |
| ZV | 5 | 4.6 | 0.74 | 4.75 | 4.25 | 5.25 | 3.5 | 5.25 |
| PL-Z | 7 | 4.57 | 0.31 | 4.5 | 4.25 | 5 | 4.25 | 5 |
| ZP | 4 | 4.5 | 0 | 4.5 | 4.5 | 4.5 | 4.5 | 4.5 |
| KV | 5 | 4.75 | 0.18 | 4.75 | 4.75 | 4.75 | 4.5 | 5 |

Interference score

| | | | | | | | | |
|---|---|---|---|---|---|---|---|---|
| Placebo | 5 | 4 | 1.87 | 4 | 4 | 5 | 1 | 6 |
| PL-L | 5 | 3.4 | 1.34 | 4 | 2 | 4 | 2 | 5 |
| PL-H | 3 | 6 | 1 | 6 | 5 | 7 | 5 | 7 |
| PL-E | 3 | 3 | 1 | 3 | 2 | 4 | 2 | 4 |
| BS-ZP | 4 | 3.75 | 2.22 | 4 | 2 | 5.5 | 1 | 6 |
| PL-Z | 7 | 4.9 | 2.1 | 5.29 | 3.57 | 5.71 | 1.29 | 8.14 |
| ZP | 4 | 4.57 | 0.87 | 4.43 | 3.93 | 5.21 | 3.71 | 5.71 |
| ZV | 5 | 4.06 | 2.14 | 5 | 1.86 | 5.29 | 1.71 | 6.43 |
| KV | 5 | 4.2 | 1.97 | 4.43 | 3.71 | 5.43 | 1.14 | 6.29 |

The study results showed that the different dose combinations of Z. officinale and V. negundo showed a dose-dependent effect on exercise-induced pain. Out of all the groups, the ZV group having V. negundo (178 mg)+Z. officinale (22 mg) had the highest number of participants achieving MPR. As compared to the Z. officinale and V. negundo group, other IPs containing P. lanceolata as their main ingredient could not show clinically relevant analgesic effect. Also, the combination of Z. officinale and P. guajava had a relatively lower impact on the outcomes.

Overall, the study results showed that the combination of Z. officinale and V. negundo in the specified dose of 22 mg and 178 mg (ZV) provides clinically meaningful pain relief in a considerably larger population compared to other groups. This product could serve as a promising lead for further trials for rapid analgesia in musculoskeletal conditions.

From the foregoing, it will be appreciated that, although specific embodiments of the invention have been described herein merely for purposes of illustration, various modifications may be made without deviating from the spirit and scope of the invention and should not be construed so as to limit the scope of the invention or the appended claims in any way.

### Advantages of Invention

Advantage of the herbal composition of the present invention is effective to alleviate chronic non-specific low back pain and related dysfunction, acute or chronic musculoskeletal pain, reducing disability, reduce joint pain and increasing work productivity.

### Abbreviation

VAS: Visual analog scale
RMQ: Roland Morris Disability Questionnaire
MPR: meaningful pain relief
BPI: brief pain inventory
CI: Confidence interval;
Max.: Maximum value of range;
Min.: Minimum value of range;
N: Number of participants
p25: 25^{th} percentile
p75: 75^{th} percentile
SD: Standard deviation

What is claimed is:

1. A herbal composition comprising of standardized Vitex negundo leaf extract and
Zingiber officinale rhizome oleoresin and a pharmaceutical acceptable carrier silicon dioxide to make carrier free composition or minimal use of carrier in a composition, wherein the ratio of Vitex negundo leaf extract to Zingiber officinale rhizome oleoresin to pharmaceutical acceptable carrier is in the range of 80:20:0 to 89:9:2.

2. The composition of claim 1, wherein the acceptable carrier is silicon dioxide only. There is no use of multiple carriers can be TWEEN 80, polysorbate, and maltodextrin, or in the combination in the range of 20-50% or more.

3. A method for treating or preventing acute or chronic musculoskeletal pain, physical-activity induced joint pain and related dysfunction comprising administering an effective amount of the herbal composition of claim 1 to a subject in need of such treatment.

4. The method of claim 3, wherein the physical-activity induced joint pain consist of mechanical loads on joints, reducing exercise-induced knee joint pain and chronic non-specific low back pain.

5. The method of claim 3, wherein the herbal composition is administered by intravenous injection, by injection into tissue, intraperitoneally, orally, or nasally.

6. The method of claim 3, wherein the herbal composition is administered orally.

7. The method of claim 3, wherein the herbal composition is in the form selected from the group consisting of a solution, dispersion, suspension, powder, capsule, tablet, pill, time release capsule, time release tablet, and time release pill.

8. The method of claim 3, wherein the effective amount of the herbal composition in the range of from about 0.001 mg per kg body weight per day to about 100 mg per kg body weight per day.

9. The method of claim 3, wherein the herbal composition is administered at least once daily.

10. The method of claim 3, wherein the subject is a mammalian subject.

11. A use of herbal composition of claim 1 to control acute or chronic musculoskeletal pain, physical-activity induced joint pain and related dysfunction.
